(19) United States
(12) Patent Application Publication
(10) Pub. No.: US 2026/0306422 A1
(43) Pub. Date: Oct. 8, 2026

(54) PERFLUOROCARBON COMPOSITIONS, SYSTEMS, AND METHODS FOR ISCHEMIC, INFLAMMATORY, INFECTUOUS, TRANSPLANT, AND OTHER MEDICAL CONDITIONS

(71) Applicant: MATTHEW T. MCLEAY, Omaha, NE (US)

(72) Inventor: MATTHEW T. MCLEAY, Omaha, NE (US)

(21) Appl. No.: 19/298,998

(22) Filed: Aug. 13, 2025

Related U.S. Application Data
(60) Provisional application No. 63/682,692, filed on Aug. 13, 2024.

(51) Int. Cl. A61K 31/02; A61K 9/00; A61K 31/198; A61K 33/00; A61K 38/44; A61P 39/06; A61P 41/00

(52) U.S. Cl. CPC A61K 31/02; A61K 9/0043; A61K 31/198; A61K 33/00; A61K 38/44; A61P 39/06; A61P 41/00

(57) ABSTRACT

Compositions for the treatment of acute manifestations because of oxidative injury by reactive oxygen species (ROS) and chronic development of fibrosis are provided. Compositions of the present invention include perfluorocarbon, hydrogen and oxygen gas, N-acetylcysteine, and glycine to counter robust oxidative responses unleashed following injury from both mechanical and non-mechanical conditions. Methods are also disclosed for delivery to challenging locations (olfactory cleft, spinal canal) and in hostile conditions (post trauma brain, spine, and upper and lower airways), with the invention's products “calming” inflamed tissue by neutralizing ROS in both acute and later complications. Active pharmaceutical ingredient(s) can also be employed to provide both acute and chronic benefit, and such active ingredients themselves can be augmented by the composition, enabling increased drug uptake and oxygenation. The present invention provides broad relief in many conditions for both human and companion subjects.

### CROSS-REFERENCE TO RELATED APPLICATIONS

This application claims priority pursuant to 35 U.S.C. 119(e) to co-pending U.S. Provisional Patent Application Ser. No. 63/682,692, filed Aug. 13, 2024, the entire disclosure of which is incorporated herein by reference.

### FIELD OF THE INVENTION

The present invention relates to perfluorocarbon compositions, manufacturing systems, and methods used for diagnosis and treatment by inhalable, topical, oral, injectable, or instilled routes for ischemic, inflammatory, infectious, transplant, and other medical conditions. More specifically, the present invention relates to inhaled dry powder compositions or inhaled or instilled liquid compositions, including perfluorocarbons such as perflubron (also known as perfluorooctyl bromide) being delivered by an intranasal or a direct approach for traumatic brain injury (TBI), spinal cord injury (SCI), acute respiratory distress syndrome (ARDS), and ischemia and/or dysfunction of procured organs for transplant (Tx), respectively. In all aspects, the compositions and manufacturing of the present invention are to be used alone or combined with a variety of therapeutics and devices such as verteporfin and ultrasound therapy.

### BACKGROUND OF THE INVENTION

The efficacy of many pharmaceutical agents is predicated on their ability to proceed to one or more selected target site(s) and remain there in effective concentrations for sufficient periods of time to accomplish a desired therapeutic or diagnostic purpose. Oftentimes, difficulty in achieving efficacy is exacerbated by the location and environment of the target site as well as by the inherent physical characteristics of the compound administered. For example, drug delivery via routes subject to repeated drainage or flushing as part of the body's natural physiological functions offer significant impediments to the effective administration of pharmaceutical agents. In this respect, delivery and retention problems are often encountered when administering compounds through the upper or lower respiratory or gastrointestinal tracts. Repeated administration of large doses is often required to compensate for the amount of drug washed away and to maintain an effective dosing regimen when employing such routes. Such reductions in delivery and retention time complicate dosing regimens, waste pharmaceutical resources, and generally reduce the overall efficacy of the administered drug.

Perfluorocarbons (PFCs), such as perflubron (PFOB), are a group of inert products with a wide range of clinical applications, from imaging to therapeutics, and PFCs have been evaluated for decades in both preclinical and clinical studies. In the art, fluorocarbon liquids have been used for treatment of respiratory distress syndrome by removal of lung debris, inflammatory cells and materials by lavage, and by facilitating oxygen delivery. In this capacity, fluorocarbons are used as neat formulations (i.e., liquid formulation with no emulsifying agents). While partial liquid ventilation using perfluorocarbons was shown to improve oxygenation and decrease lung injury in various animal models, clinical trials concluded that neither a high dose nor low dose of perfluorocarbon improved outcome in patients with respiratory distress (See Kacmarek R M, et al. AM J Respir Crit Care Med. 2006 Apr. 15; 173(8):882-9). The large volume and shear weight of perflubron appears likely to have contributed to this failed strategy.

Further studies include testing of PFCs as a diagnostic (See MRI Imagent GI, Echocardiogram Definity), therapeutics such as a “blood substitute,” an organ preservative (2-layer method for pancreas), and a treatment for ARDS in the form of “liquid ventilation.” Although these developments have had variable successes, the studies have been limited in scope and lacking in diagnostic and therapeutic application to conditions with high unmet medical needs, such as traumatic brain injury (TBI), spinal cord injury (SCI), acute respiratory distress syndrome (ARDS), and transplant ischemia and dysfunction. It would be beneficial to have compositions and devices to provide improved diagnostics and therapeutics. Furthermore, it would be beneficial to have a composition, system, and method for effective treatment for acute and chronic traumatic brain injury (TBI), spinal cord injury (SCI), acute respiratory distress syndrome (ARDS), and transplant (Tx) preservation, among other medical conditions and diseases.

A common pathological finding among these conditions is a marked increase in reactive oxygen species (ROS) resulting in inflammation and ultimately cell death. ROS includes free radical species such as singlet oxygen, hydroxyl radical, and superoxide and non-radical species such as hydrogen peroxide and peroxynitrite. Each of these can disrupt critical biochemical reactions, organelles, DNA, proteins, and lipids. Some ROS can be neutralized by cellular glutathione, superoxide dismutase, catalase and other antioxidants such as hydrogen gas. (See Rauf et al. (2024); Reactive oxygen species in biological systems: Pathways, associated diseases, and potential inhibitors—A review. Food Science & Nutrition, 12, 675-693; https://doi.org/10.1002/fsn3.3784).

Nie et al. used an easily accessible intravenous route in mice, generally assuring a perflubron (PFOB) and hydrogen (H₂) nanoemulsion would interface with its target ROS, and indeed PFOB was taken up within 1 hour during reperfusion of the single obstructed left coronary artery. In Nie et al., PFOB-H₂ was seen in Cd11b macrophages within 1 hour after intravenous administration. In contrast, an intranasal topical application for traumatic brain injury (TBI) encounters a far more heterogenous path to the site of pathology as compared to intravenous application. Furthermore, Nie et al.'s 1 hour ischemia is also brief compared to the protracted and variable ischemia of TBI, SCI, ARDS and a Tx organ's injury. (See Nie et al., Controlled Release of Hydrogen-Carrying Perfluorocarbons for Ischemia Myocardium-Targeting ¹⁹F MRI-Guided Reperfusion Injury Therapy. Adv. Sci. 2023, 10, 2304178; https://doi.org/10.1002/advs.202304178).

Similarly, Tamura et al. 2024 used inhaled H₂ (but no PFC) along with treatment for mild hypothermia, post cardiac arrest, and while there was improved neurological outcomes, there was no improvement in survival. (See Tamura et al. ; Combination of Hydrogen Inhalation and Hypothermic Temperature Control After Out-of-Hospital Cardiac Arrest: A Post hoc Analysis of the Efficacy of Inhaled Hydrogen on Neurologic Outcome Following Brain Ischemia During PostCardiac Arrest Care II Trial; Crit Care Med. 2024 Oct. 1;52(10):1567-1576; doi:10.1097/CCM.0000000000006395).

Multiple antioxidants have been utilized clinically, and supplying precursors can benefit cells, protecting them from cell death. For example, n-acetylcysteine (NAC) coupled with glycine enables adequate intracellular glutathione, and the addition of hydrogen gas can also reduce ROS.

Nevertheless, traumatic brain injury (TBI) studies have not demonstrated clear efficacy with single antioxidants alone (See Fesharaki-Zadeh, A. (2022). Oxidative Stress in Traumatic Brain Injury. International Journal of Molecular Sciences, 23(21), 13000; https://doi.org/10.3390/ijms232113000). This emphasizes a critical need for both H₂ and NAC/Gly for glutathione (and future catalase) since neither H₂ nor glutathione can resolve the ROS injury alone. For example, H₂ gas neutralizes hydroxyl and peroxynitrite, whereas NAC/Gly for glutathione neutralizes hydrogen peroxide as a co-factor to glutathione peroxidase by donating hydrogen. After SOD dismutates superoxide, that becomes hydrogen peroxide, glutathione then also becomes responsible for resolving superoxide as well. The future addition of catalase directly impacts hydrogen peroxide.

No PFC has been clinically approved for use in the human upper or lower airway, but large volume liquid perflubron was used in a phase II/III study for adult respiratory distress syndrome (ARDS), instilling many liters of perflubron into the airway of patients on mechanical ventilation with very severe inflammation. Overall, the mortality was unchanged but hypoxic and hypotensive episodes (presumed due to ventilator disconnection) and increased pneumothorax were observed in the perflubron group with 28% perflubron vs. 9% control. This was probably due to the “heavy” perflubron which has a density nearly twice that of water and created overdistension (See Kacmarek et al. ; Partial liquid ventilation in adult patients with acute respiratory distress syndrome; Am J Respir Crit Care Med. 2006 Apr. 15;173(8):882-9. doi:10.1164/rccm.200508-11960C).

In contrast, small volume nebulization of neat perflubron in animals has shown to increase oxygenation in a neonatal swine model and some anti-inflammatory benefits have been suggested (See Kandler et al. ; Dose Response to Aerosolized Perflubron in a Neonatal Swine Model of Lung Injury. Pediatr Res 56, 191-197 (2004);

https://doi.org/10.1203/01.PDR.0000132667.47744.F4), but no airway delivery of perflubron or other PFCs with specific gas-based recipes has been used alone or in combination. Dry powder inhalers have also been used in therapy, but PFCs have been removed by spray drying in a terminal step to remove, e.g., perflubron, to create porous structures containing crystal active pharmaceutical ingredient(s) (API) and phospholipids, sometimes referred to as “pulmospheres.” (See Med Devices (Auckl). 2011; 4: 179-188; Respir Res. 2023; 24: 96) (See also Pharmaceutical Particle Engineering via Spray Drying, 2008).

Manufacturing techniques for dpi compositions using perflubron teach against retaining perflubron, as removal leaves porous surfaces. “The decreased interparticle cohesive forces achieved with spray-dried powders, particularly those with highly porous surfaces, leads to improvements in lung delivery efficiencies, with up to 60% of the nominal dose delivered to the lungs.” (See Mohan et al. ; Advancements in the Design and Development of Dry Powder Inhalers and Potential Implications for Generic Development. Pharmaceutics. 2022 Nov. 17;14(11):2495. doi: 10.3390/pharmaceutics14112495. PMID: 36432683; PMCID: PMC9695470). Perflubron spray drying is thought to “fly” with a less dense, “whiffle ball” like molecules and create less agglomeration (see

https://www.tobipodhalerhcp.com/About/PULMOSPHERE-Technology).

A potential limitation of dpi delivery is the hostile environment within both the upper intranasal and lower airways and at the alveolar lining fluid, with significant inflammatory products, mucus, and structural changes, among others, such as with COPD/emphysema, asthma, and pulmonary fibrosis. Nevertheless, retained perflubron may provide significant advantages, and heretofore there has not been available a dry powder inhaler (dpi) composition, manufacturing system, or method for retaining perflubron within dpi compositions with the advantages and features of the present invention.

PFCs have also been used in both preclinical and clinical studies for management of blood loss with demonstrable benefits of transfusion avoidance. A successful Phase III study was accomplished using perflubron emulsion as a “blood substitute,” however a follow-on Phase III study had an imbalance of strokes, which was ultimately concluded to be due to protocol choices and protocol violations (See Krafft M P, Riess J G (2021); Therapeutic oxygen delivery by perfluorocarbon-based colloids. Adv Colloid Interface Sci 294:102407). In an unpublished, single patient emergency investigational new drug study (IND), a 60% perflubron emulsion was nebulized 0-50 mL daily for 1 week (a 25 mL dosage was used when administered), and remarkable x-ray changes (see FIG. 3) and lung mechanics were observed in the first 9 hours following administration. The rapid improvement was felt to be due to mucus and inflammatory debris clearing with perflubron “getting underneath” the inflamed airway lining fluid, increasing oxygenation and reduced mechanical ventilation needs, confirming pre-clinical studies (Kandler et al. ; 2004 Pediatric Research 56 191-197). Further inflammation signals, e.g., C-reactive protein, correlated with nebulization administration.

Based on the foregoing, a need exists for manufacturing therapeutic delivery vehicles with improved efficacy and target site retention while at the same time minimizing dosage. Also needed is a therapeutic capable of targeting tissue-resident cells. The manufacturing and compositions and methods of the present invention provide therapeutics and therapeutic delivery vehicles with improved efficacy and target site retention while minimizing toxicity.

Heretofore there has not been available perfluorocarbon compositions, systems, or methods with the advantages and features of the present invention.

### SUMMARY OF THE INVENTION

The present invention comprises compositions, systems, devices, methods of manufacture, and methods of use which retain one or more perfluorocarbons (PFCs) along with one or more active pharmaceutical ingredients (APIs), resulting in a theranostic clinical product. In an exemplary embodiment, a PFC of the present invention comprises perflubron (PFOB). In an exemplary embodiment, the manufacturing steps of perfluorocarbon retention benefit a wide range of API compositions plus excipients and emulsions.

In an exemplary embodiment, the present invention uses a novel composition and method to manage reactive oxygen species (ROS) by first incorporation of both perfluorocarbons (PFC) with a hydrogen and oxygen gas mixture, plus acetylcysteine and glycine (and later catalase). In some embodiments, such compositions are coupled with specific methods of delivery and devices, including ultrasound for both drug uptake in acute inflammation and scar clearance in the later chronic manifestations of the conditions.

In embodiments, N-acetylcysteine (NAC) and glycine (and future catalase) are utilized in ROS neutralization, particularly in conditions when facing a daunting trek to the brain, spinal cord, lung, and transplant organs. Although transplant (Tx) applications use the intravascular “roadways,” the present Tx invention does not rely on a “blood” requirement which is in effect a component of the composition contributing antioxidants.

In embodiments, the present invention utilizes the addition of precursor glutathione products, NAC and Glycine (and future catalase).

In embodiments, the combination of antioxidants of the present compositions provide full coverage of ROS using PFOB-H₂—O₂—NAC Glycine, even in hypothermia, since, unlike blood, O₂ release from PFOB is not impacted by cold temperatures.

Notably, the present invention uses combinations of antioxidants and novel delivery to augment transfer of the products to reduce the impact of ROS. In traumatic brain injury embodiments, efficacy of the invention occurs based on perflubron's high spreading coefficient and teaches the simple creation of a reservoir, e.g., the olfactory cleft, by placing the patient is in supine position so as to take advantage of perflubron's density, hydrophobicity, and solubility of H₂ as compared to water. Further, this coupled with the use of acetylcysteine and glycine (and later catalase) leads to replenishing intracellular glutathione and neutralizing ROS. Such provides advantages over prior art compositions and methods. By way of example, the composition of PFOB-H₂ disclosed by Nie et al. would not provide such features because H₂ does not neutralize hydrogen peroxide, so there is an unprotected effect against ROS cell death.

Embodiments of the present invention include liquid, foam, hydrogel, and dry powder inhaler (dpi) compositions, for intranasal and/or oral and airway delivery, manufactured with a unique method of retaining perflubron, with or without excipient or a liquid emulsion, with or without gas-based loading of the PFC with hydrogen and oxygen gas, acetylcysteine, and glycine.

In embodiments, intranasal approaches envision the use of liquids, dry powder inhaler, foam or hydrogels, among others, with gas and drug additions that provide exceptional treatment for trauma, whether as a result of TBI, SCI, or ARDS. Non-trauma applications using an intranasal approach include use for post-transplant dysfunction. The present invention provides for, among others, effective and efficient applications for soldiers, civilians, and transplant recipients, possibly alleviating decades of disability.

In some embodiments, ultrasound is used to both increase delivery of the composition by release and also provide impact on epithelial borders.

With respect to dpi compositions, the present invention provides a novel approach to the conventional practice of eliminating perflubron in a second spray drying manufacturing step (See Mohan et al. ; Pharmaceutics 2022, 14(11), 2495;

https://doi.org/10.3390/pharmaceutics14112495). The present invention results in a manufactured product that is a fluorochemical composition whose clinical application improves delivery of bioactive agents. The present invention teaches that the intentional retention of perflubron in dpi compositions improves most clinical conditions that currently employ dpi. In further embodiments of the present invention, new dpi compositions of the present invention include active pharmaceutical ingredients (APIs), such as monoclonal antibodies (Mab), given the protection a perflubron emulsion affords a nebulized Mab. Moreover, the present invention provides an improved manufacturing process by eliminating a second spray dry step and reduced perflubron collection requirements. The addition of combination gases follows delivery of the dpi product, whereas such would be incorporated into a liquid aerosol or instilled topical administration.

Embodiments of the present invention employ perflubron in a variety of applications, including direct injection taking advantage of perflubron's unique and significant characteristics, including a high spreading coefficient, facilitation of oxygen delivery, and anti-inflammatory properties. An emulsion and/or other excipients are also utilized with perflubron improve overall drug delivery. In an exemplary embodiment, the delivery of a fragile drug, exosome, or large proteins such as monoclonal antibodies (Mab) result in a markedly increased uptake of the active ingredient. In embodiments, a diagnostic product is benefited by being escorted through the human upper or lower airway and lining fluid inside a perflubron emulsion. Further, an API receives the benefit of perflubron's hydrophobic spreading properties while perflubron also mitigates frequently encountered hypoxic areas found in cancer and non-cancer locations.

Many drugs, such as, but not limited to gemcitabine, are sensitive to degradation and hypoxia. Nevertheless, the presence of a perflubron emulsion enables a hydrophilic drug such as gemcitabine to be delivered in the hydrophilic portion of the emulsion, presumably protecting against enzymes such as cytidine deaminases and enabling a steady release.

Additionally, the predominant fluorine molecule in perflubron, and its absence in humans naturally, further makes the addition of perflubron a possible imaging advantage.

Fluorine can be captured using 19 FMRI/spectroscopy and bromine using CT/fluoroscopy/x-ray imaging. Embodiments of the present invention may also incorporate molecules such as sensitizers, including but not limited to IRDye800CW, to monoclonal antibodies such as checkpoint inhibitors (e.g., pembrolizumab) or intrinsically fluorescent products like verteporfin in the final composition. Coupled with perflubron, the present compositions, and manufacturing methods thereof provide not only imaging advantages (as discussed above) but also therapeutic applications for a wide range of conditions including TBI, SCI, ARDS and transplants and could be coupled with devices such as photodynamic, sonodynamic, and radiodynamic therapy especially during chronic manifestations of these conditions.

In further aspects of the present invention, exhaled nitric oxide (eNO) is measured in a patient to help measure, determine, and/or predict the severity of reactive oxygen species (ROS) present in the patient as a result of medical conditions, such as TBI, SCI, ARDS and transplants.

The foregoing and other objects are intended to be illustrative of the invention and are not meant in a limiting sense. Many possible embodiments of the invention may be made and will be readily evident upon a study of the following specification and accompanying drawings comprising a part thereof. Various features and subcombinations of invention may be employed without reference to other features and subcombinations. Other objects and advantages of this invention will become apparent from the following description taken in connection with the accompanying drawings, wherein is set forth by way of illustration and example, an embodiment of this invention and various features thereof.

### BRIEF DESCRIPTION OF THE DRAWINGS

Embodiments of the present invention are described in detail below with reference to the attached drawing figures, wherein:

FIG. 1 shows an image created using the large specimen imaged with a non-contrast CT from a patient with metastatic ovarian cancer, with the white areas demonstrating radiopaque perflubron scattered throughout the omentum.

FIG. 2 shows images illustrating the benefit of a perflubron emulsion-monoclonal antibody composition (such as Mab trastuzumab) with markedly increased binding as compared to water with no perflubron emulsion.

FIG. 3 shows x-ray images depicting before and after perflubron emulsion nebulization was given to a patient with ARDS.

FIG. 4 shows enlarged image comparison of “pulmospheres,” which are dpi particles with spray dry evaporation, leaving open “holes” where perflubron was present, and dpi particles with retained perflubron.

FIG. 5 shows intranasal administration of a composition of the present invention toward the olfactory epithelium of a patient.

FIG. 6 shows treatment and exhaled nitric oxide (eNO) measurement schedule and drug treatment groups and control for experimental testing example showing correlation of eNO and viral load.

FIGS. 7-13 show graphical data illustrating correlation of eNO and viral load. Graphical data shows 20 minute data of eNO, reproducibility of eNO, effect of superoxide dismutase (SOD) on eNO, effect of aminoguanidine on eNO, and graphical representation of correlation between H5NI viral load and eNO.

The drawing figures do not limit the present invention to the specific embodiments disclosed and described herein. The drawings are not necessarily to scale, emphasis instead being placed upon illustrating the principles of the invention.

### DETAILED DESCRIPTION OF THE PREFERRED EMBODIMENTS

As required, a detailed embodiment of the present invention is disclosed herein; however, it is to be understood that the disclosed embodiment is merely exemplary of the principles of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

In exemplary embodiments of the present invention, manufactured fluorochemical compositions are contemplated as directly injected, instilled aerosols, liquids, and/or dry powder inhaler (dpi) compositions.

For dpi, one skilled in the art will appreciate that the present invention creates routes and methods of administration for the intended use of the compositions, accounting for the location of the target area, and the condition being treated, in addition to other factors known in the art such as subject health, age, and physiological status.

Disclosed are manufacturing and pharmaceutical formulations for instilled, aerosol, injected, and dry powder inhalers intended for intranasal and oral airway routes. In embodiments, compositions of the present invention include neat perfluorocarbon (PFC), either alone or in combination with gases such as molecular hydrogen (H₂) and molecular oxygen (O₂). In some embodiments, compositions of the present invention include N-acetylcysteine (NAC) and/or glycine in combination with neat or emulsion PFC, such as but not limited to perflubron, with excipients or stable micellar suspension. In another embodiment, the perflubron is combined with one or more monoclonal antibody(ies) (Mab), exosomes, sonosensitizers, and/or traditional drugs, among others. In embodiments, gases within compositions of the present invention maybe dissolved or delivered by cannula or mask that the PFC will dissolve. In some embodiments of intranasal administration of the present invention, the intranasal administration is performed with the patient in supine position. In other embodiments, the intranasal administration is performed with the patient in an upright or sitting position.

Perflubron is hydrophobic, mildly lipophilic, and at room air is a liquid. In exemplary embodiments of the present invention, compositions include perflubron plus hydrogen dissolved in the perflubron. In embodiments, perflubron compositions, either with or without hydrogen, further include other antioxidants, such as but not limited to NAC, derivative NAC-amide, plus, in further embodiments, glycine and/or catalase.

In exemplary embodiments, a perflubron-retained product may contain one or more photosensitizers including, but not limited to, IRDye 700DX or 800CW, aminolevulinic acid (ALA), indocyanine green (ICG), verteporfin, or other conjugated products bonded to monoclonal antibodies, affibodies, proteins, peptides, exosomes, and/or nucleic acids. In some embodiments of the present invention, an airway upper or lower delivery has significant advantages over an intravenous route including reduced dosing because of direct interface with affected tissue, no first pass metabolism through the liver or other organs, decreased side effects due to direct dosing at the site of the condition, improved imaging due to the higher concentration and improved tumor-to-background ratio possible by direct application, and/or the ease of repeat dosing.

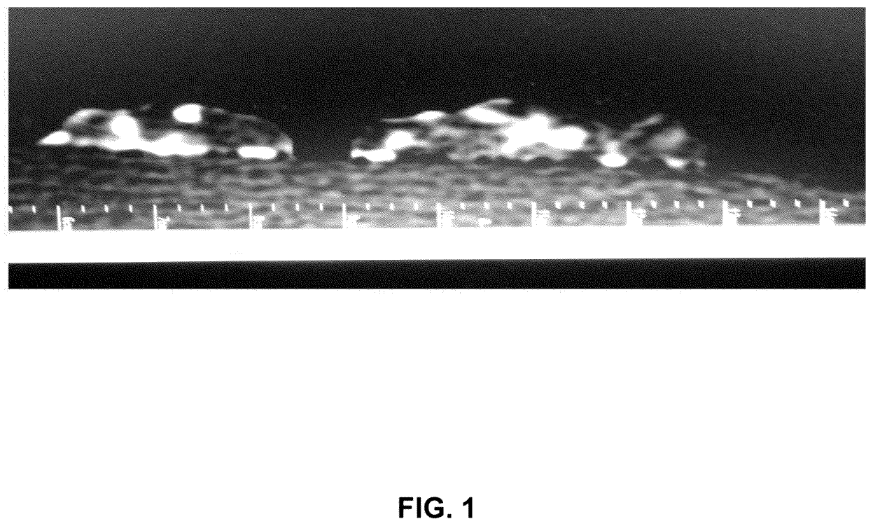

Referring to the drawings in more detail, FIG. 1 demonstrates one aspect of why perflubron retention during manufacturing is beneficial, including for quality measures, as perflubron enables visualization of the product by a variety of modalities, such as x-ray, CT, US/PAI, MRI, or Raman, and other spectroscopic measurements. FIG. 1 depicts a large omentum tissue sample with a human female cancer. The image was created using the large specimen imaged with a non-contrast CT from a patient with metastatic ovarian cancer. The tissue was submerged in a 60% perflubron emulsion for 3 hours at 37 degrees Fahrenheit and placed on a rocker bed. The white areas of the image demonstrate radiopaque perflubron scattered throughout the omentum.

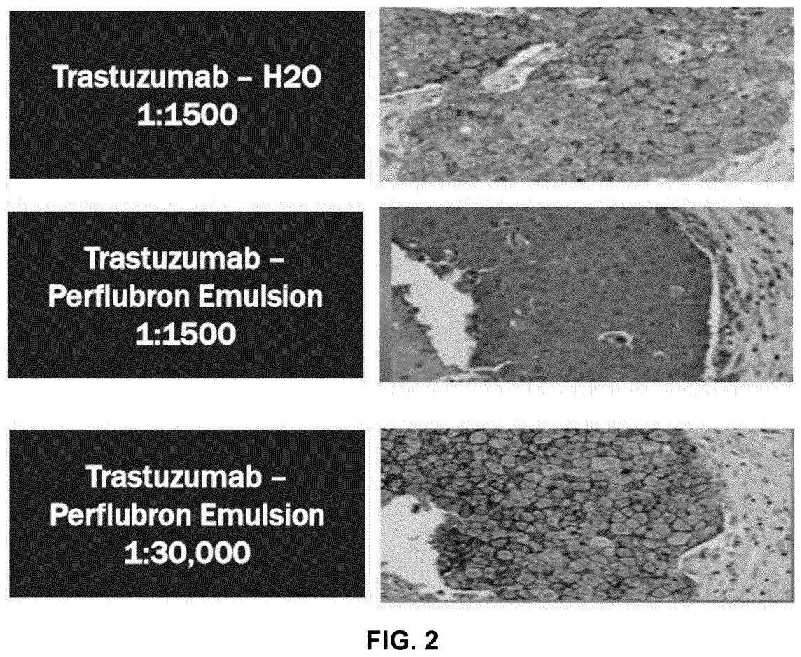

FIG. 2 illustrates the benefit of a perflubron emulsion-monoclonal antibody composition (such as Mab trastuzumab) with markedly increased binding. The present invention contemplates how delivery of a Mab can be enhanced via multiple routes including dpi by altering manufacturing to retain both water and perflubron. Such compositions have significant clinical benefits while also enabling quality evaluations and use of lower doses of API, thus reducing exposure and cost. In further embodiments, other monoclonals, such as checkpoint inhibitors like pembrolizumab, similarly within perflubron emulsion with augmented delivery and response. FIG. 2 further shows the nebulized product in water also displayed more fracturing on SDS-PAGE, supporting that perflubron emulsion protects and cloaks the API during aerosol delivery.

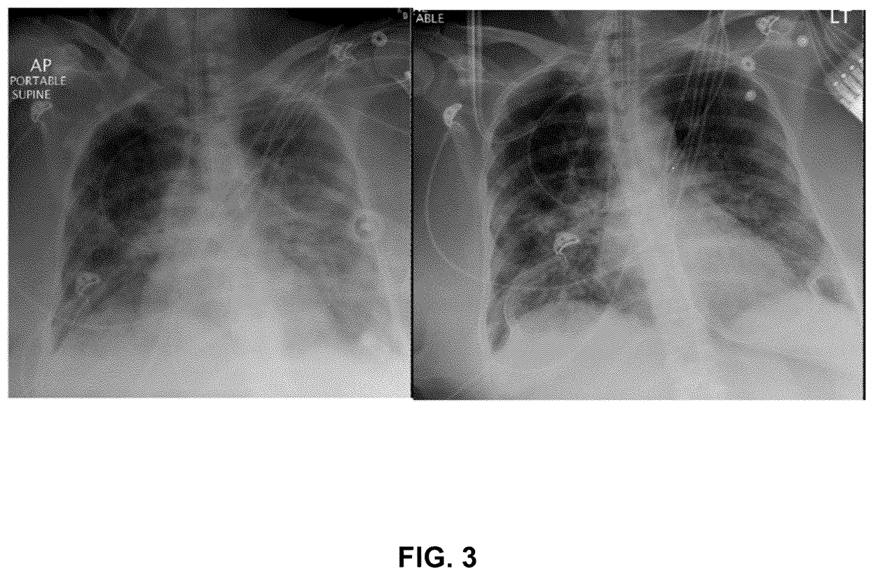

FIG. 3 shows perflubron emulsion nebulization given to a patient with ARDS.

The x-ray images shown in FIG. 3 demonstrate why perflubron emulsion retention in dpi manufacturing is a significant advantage for clearing airway mucus and debris and reducing inflammation, presumably mitigating fibrosis. The x-rays show a nebulized perflubron emulsion administration demonstrated dramatic rapid clinical/radiographic changes in 9 hours and durable over 1 week in a patient with severe H1N1 ARDS. No other changes in medications for this patient during the 9 hours account for such improvement. In embodiments, perflubron or perflubron emulsion in compositions of the present invention will clear or lift up the mucus layer when a patient is laying down, the perflubron will interface with the olfactory epithelium, lift the water layer and mucus up and off the surface, and clear the mucus from nasal cavity, just like in lung x-rays showing much less white patchy areas after administration.

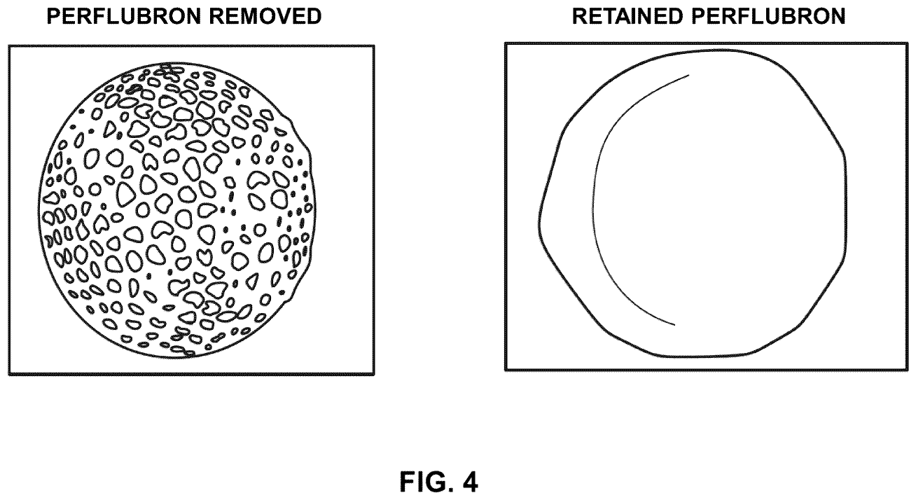

FIG. 4 shows a comparison of the (enlarged) appearance of dpi particles with retained perflubron and the (enlarged) appearance of “pulmospheres,” or dpi particles formed with conventional spray dry evaporation, leaving open “holes” where perflubron was present. In the conventional dpi manufacturing process, a two-stage drying process is utilized: first, a heating and droplet drying process is utilized to remove water, and then, a spray drying process is utilized which further heats and removes perflubron, creating particles with holes where perflubron was present. In contrast, the present invention does not utilize such a second step and regains perflubron within the dpi particles.

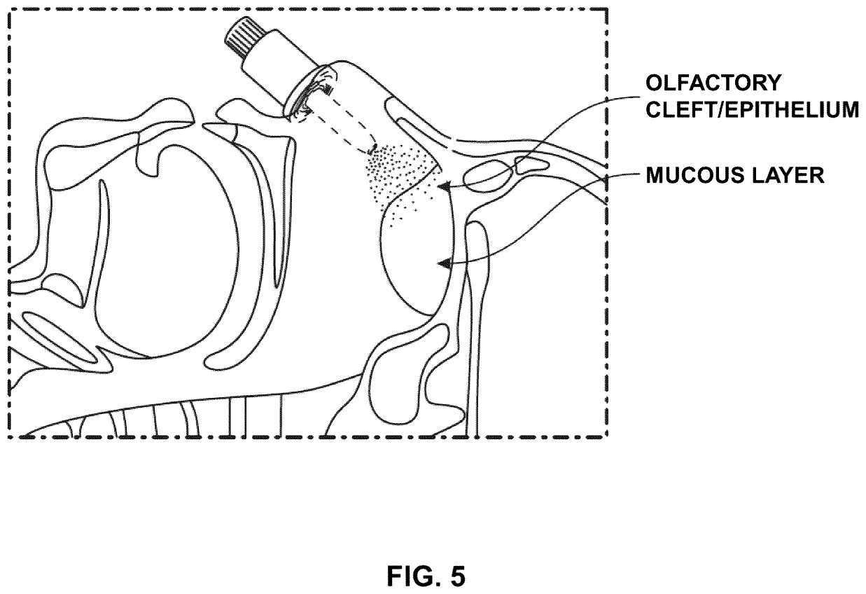

FIG. 5 shows a diagram illustrating an embodiment of intranasal delivery of compositions of the present invention. In embodiments, when perflubron is delivered to the lower airway, mucus is lifted away, including in the nasal upper airway where the intention is to clear the mucus layer at the olfactory epithelium. In embodiments, if perflubron is further carrying an active pharmaceutical ingredient, it will be more easily delivered because the olfactory mucus generally guards products from getting in brain. In embodiments, after intranasal administration, the perflubron sits in the olfactory cleft of the patient and further continually releases O₂ and H₂ (and/or other API) over time. If water was in the olfactory cleft instead of perflubron, there would be minimal gas dissolved, but perflubron provides a gas reservoir, and the brain needs H₂ and O₂ after a trauma.

In an embodiment, an intranasal delivery facilitates delivery for intracranial applications. In an exemplary embodiment, a perfluorocarbon composition with the addition of hydrogen and oxygen gases plus NAC and glycine is configured to be promptly taken up by the olfactory cleft epithelium. Such composition is far more efficiently delivered as compared to simple delivery in water or even lipophilic products that do not contain a perfluorocarbon such as perflubron. Unlike many other applications of active pharmaceutical ingredient(s) (APIs), exemplary embodiments of the present invention do not require special equipment, a hospital, or specialized care, some or all of which is most often required for intravenous, intrathecal, or intraoperative administration of APIs. Such delivery methods of embodiments of the present invention allows for use in both remote locations, such as on a battlefield or in community areas in the case of treatment of traumatic brain injury.

In an embodiment, a manufactured formulation of the present invention combines perflubron with anti-fibrotic medications, such as but not limited to nintedanib or pirfenidone, either alone or in combination.

In embodiments, manufactured formulations of the present invention combine perflubron with pharmaceuticals that are configured for delivery intranasally to the brain or into the lung from a nasal or oral approach and enter systemic circulation.

In embodiments, manufactured dry powder perflubron-containing formulations include ribavirin.

In embodiments, manufactured dry powder perflubron-containing formulations include gemcitabine.

In embodiments, manufactured dry powder perflubron-containing formulations include verteporfin.

In further embodiments, dry powder perflubron-containing formulations of the present invention include a combination of ribavirin, gemcitabine, and verteporfin; a combination of ribavirin and gemcitabine; a combination of ribavirin and verteporfin; or a combination of gemcitabine and verteporfin.

In embodiments, manufactured dry powder perflubron-containing formulations include monoclonal antibody(ies) (Mabs), such as but not limited to pembrolizumab (commonly referred to as Keytruda) attached to a photosensitizer and/or sonosensitizer.

In embodiments, manufactured dry powder perflubron-containing formulations\include a combination of multiple active pharmaceutical ingredients (APIs).

In embodiments, formulations of the present invention include an excipient. In exemplary embodiments, the excipient is one or both of fumaryl diketopiperazine and a sugar alcohol, such as mannitol.

In exemplary embodiments, dry powder formulations of the present invention contain 96% perflubron, an API, and emulsion/excipient and no more than 4% water. In embodiments, dry powder formulations of the present invention contain 10% or less, 9% or less, 8% or less, 7% or less, 6% or less, 5% or less, 4% or less, 3% or less, 2% or less, 1% or less, or 0% water. In embodiments, dry powder formulations of the present invention contain 90% to 99.9%, 90%, 91%, 92%, 93%, 94%, 95%, 96%, 97%, 98%, 99%, 99.9%, about 90%, about 91%, about 92%, about 93%, about 94%, about 95%, about 96%, about 97%, about 98%, about 99%, or 99.9% perflubron, API, and emulsion/excipient.

In embodiments, monoclonal antibodies (Mabs) or exosomes, both to treat cancer and non-cancers, are utilized in formulations of the present invention to be delivered as an intranasal or oral dry powder, which formulations remain stable and therapeutically, physiologically, and/or biologically stable at room temperature for many months. In such embodiments, the powder comprises a plurality of particles, most of which are perflubron, an active agent (API), less than 6% (w/w) water, and additional ingredients that include sugar and/or a sugar alcohol, a buffer, an amino acid, and/or a surfactant. In embodiments, the particles comprise drugs, Mab, exosomes or other active agents, and less than or equal to 6% water (w/w). In some embodiments, the water in the formulation is <3% (w/w).

In embodiments, the shape of some or each particle(s) are/is spherical, approximately spherical, spheroidal, or approximately spheroidal. In further embodiments, the shape of some or each particle(s) are/is cylindrical or approximately cylindrical. In embodiments, the aspect ratio of each particle is >0.40, >0.50, >0.60, >0.70, >0.80, >0.90, or >1.00. In some embodiments, the aspect ratio of the particle is about 0.90 to about 0.98. The shape of the particle may be determined inter alia by micro-flow imaging (MFI).

In an embodiment, the mean diameter of the particle(s) is/are <50 microns. In an embodiment, the mean diameter of the particle(s) is/are <25 microns. In another embodiment, the mean diameter of the particle(s) is/are <0 to 10 microns. In further embodiments, the diameter of the particle(s) is/are 0.05 microns to 25 microns, 0.1 microns to 10 microns, 0.5 microns to 5 microns, 1 micron, 2 microns, 3 microns, 4 microns, 5 microns, about 0.05 microns to about 25 microns, about 0.1 microns to about 10 microns, about 0.5 microns to about 5 microns, about 1 micron, about 2 microns, about 3 microns, about 4 microns, or about 5 microns. Diameter may be determined inter alia, e.g., by micro-flow imaging (MFI) or static light scattering.

In embodiments, the particles(s) comprise a small molecule excipient (SME) capable of assembling into a macromolecular assembly. In some embodiment, the small molecule excipient is fumaryl diketopiperazine (FDKP). In further embodiments, the SME is present at one part SME per ten parts active agent (1:10) by weight, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, about 1:10, about 1:9, about 1:8, about 1:7, about 1:6, about 1:5, about 1:4, about 1:3, about 1:2, about 1:1, about 2:1, about 3:1, about 4:1, about 5:1, about 6:1, about 7:1, about 8:1, about 9:1, or about 10:1 parts SME to active agent, such as but not limited to verteporfin, ribavirin, and/or gemcitabine, by weight.

In some embodiments, the particle(s) also comprises a buffer, such as but not limited to phosphate, histidine, and/or acetate. In embodiments, the buffer is present at 1%, 2%, 1% to 2%, about 1%, about 2%, or about 1% to 2% (w/w).

In embodiments, the particle(s) also comprise a thermal stabilizer, which can be, inter alia, sugar, sugar alcohol, amino acid, and/or combinations thereof. Amino acids include, inter alia, alanine, isoleucine, proline, valine, leucine, and/or arginine. In embodiments, the thermal stabilizer includes trehalose, mannitol, sucrose, and/or isoleucine. In an exemplary embodiment, the thermal stabilizer is mannitol. In further embodiments, the thermal stabilizer comprises mannitol or other sugar alcohol at one part sugar alcohol per ten parts active agent (1:10) by weight, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, about 1:10, about 1:9, about 1:8, about 1:7, about 1:6, about 1:5, about 1:4, about 1:3, about 1:2, about 1:1, about 2:1, about 3:1, about 4:1, about 5:1, about 6:1, about 7:1, about 8:1, about 9:1, or about 10:1 parts sugar alcohol to active agent by weight.

In embodiments, the particle(s) also comprises a surfactant. In embodiments, the surfactant is an amphipathic nonionic surfactant, such as but not limited to a fatty acid ester of a polyoxymethylene sorbitan. In embodiments, the surfactant comprises polysorbate 20 or polysorbate 80, at 0.015-1-part polysorbate per five (5) parts by weight or about 0.015-1-part polysorbate per five (5) parts by weight. In embodiments, the polysorbate 20 or polysorbate 80 is present at 0.15, 0.3, 0.6, 1, 1.5, 3, 6, 10, about 0.15, about 0.3, about 0.6, about 1, about 1.5, about 3, about 6, or about 10 parts of polysorbate per 50 parts by weight.

In further embodiments, manufactured formulations of the present invention include liquids or dpi containing photosensitizers and/or fluorescent products will be used in the manufacturing process. In embodiments, the invention takes advantage of perflubron in the final product enabling Raman, MRI, spectroscopy, or other imaging methods to aid the manufacturing process given the specific fluorine identification of perflubron.

In a further aspect of the present invention, treatment methods further include monitoring the presence and ongoing creation of reactive oxygen species (ROS) in addition to addressing resolution of ROS. The present invention further teaches that exhaled nitric oxide (eNO) may be used to predict ROS because of the “consumption” of eNO when in the presence of superoxide that creates peroxynitrite and cellular damage, such as what occurs with TBI, ARDS, etc. When superoxide is present, eNO levels fall, but when superoxide is no longer excessively produced or when neutralized by the countermeasures of the present invention, eNO returns to normal levels. Commercial devices or any method that measure eNO, e.g., intranasal and oral airway or other routes or blood/fluids, are anticipated to assist in treatment duration management of the present invention. In an embodiment, the level of eNO is measured by an electrochemical analyzer (such as, but not limited to, the NIOX VERO® electrochemical eNO analyzer, Circassia Pharmaceuticals Company, Oxford, UK) according to American Thoracic Society (ATS) guidelines (see Ralph et al., Impaired pulmonary nitric oxide bioavailability in pulmonary tuberculosis: association with disease severity and delayed mycobacterial clearance with treatment, J Infect Dis 2013; 208:616-626).

The invention provides manufacturing of several formulations, treatment regimens, and routes of administration of active agents, including oral and intranasal liquid topical or dpi administration.

In one aspect, the present invention provides a liquid or dpi perflubron-API composition by oral or intranasal dosing that finds systemic delivery benefit in treating nasal, brain, oral, pancreatic cancer, esophageal, gastric, colon, and other gastrointestinal cancers, dysplasias, neoplasias, or hyperplasias, such as for example Barrett's esophagus, head and neck cancer, lung cancer, and non-cancer-oral leukoplakia.

In another aspect, the invention provides a perflubron liquid, foam, or hydrogel combined with gas, such as hydrogen and/or oxygen, or solid dry powder form or aerosol form of perflubron with NAC, glycine, and catalase for use in treating traumatic brain injury (TBI), spinal cord injury (SCI), acute respiratory distress syndrome (ARDS), and transplant (Tx) preservation. ARDS may be the result of lung infection, pneumonia, COPD, another acute respiratory distress syndrome, or the like.

In an embodiment, the NAC is in a dry powder that can be administered with a dry powder inhaler, such as but not limited to a breath-powered single-use or reusable inhaler. See U.S. Pat. Nos. 7,464,706B2; 8,146,588B2; WO2009121020A1; US20170216538A1; US20180228987A1; U.S. Pat. Nos. 9,700,690B2; D635241S1, WO2014144895A1, and U.S. Pat. No. D605753S1 for descriptions of exemplar breath-powered inhaler devices.

In another aspect, the invention provides a pharmaceutical formulation containing a NAC formulation of the previous aspect combined with another medicament or excipient. In an embodiment, another medicament or excipient is perfluorocarbon, such as perflubron.

In another aspect, the invention provides an airway derived source of liposomal or stable emulsion formula for use as a topical medicament for treating skin conditions such as non-melanoma skin cancer, melanoma, and chronic skin conditions such as but not limited to psoriasis.

In an exemplary embodiment, a composition for direct delivery is provided comprising 2 milliliters of perflubron, 2.5% (w/w) of hydrogen gas, 21% (w/w) of oxygen gas, 200 mg/mL of N-acetylcysteine amide, and 200 mg/mL of glycine.

In embodiments, a composition of the present invention includes 0.1 mL to 20 mL of perflubron.

In embodiments, a composition of the present invention includes 0.5% to 4% (w/w) hydrogen gas.

In embodiments, a composition of the present invention includes 10% to 96% (w/w) oxygen.

In embodiments, a composition of the present invention includes 100 mg/mL to 600 mg/mL N-acetylcysteine amide.

In embodiments, a composition of the present invention includes 100 mg/mL to 600 mg/mL glycine.

In embodiments, a composition of the present invention is configured for application to each nostril of a patient, one after the other.

In embodiments, a composition of the present invention further includes catalase (bovine liver GRAS), either alone or in an exosome, egg yolk, polysorbate emulsion.

In embodiments, compositions of the present invention include dry powder, liquid, foam, aerosol, hydrogel, or emulsion (including an emulsifier or emulsifying agent) compositions.

In embodiments, compositions of the present invention are configured to be delivered by a syringe, inhaler, or spray catheter.

In embodiments, compositions of the present invention are further configured for use with ultrasound (augment delivery of products for acute patient condition).

In embodiments, compositions of the present invention further include high molecular weight hyaluronan, monoclonal antibodies, tyrosine kinase inhibitors, nintedanib, pirfenidone, siRNA, DNA using exosomes or similar vehicles containing drugs or genetic material, or combinations thereof.

In embodiments, a method of treating acute traumatic brain injury (TBI) comprises delivering a composition of the present invention using supine positioning enabling delivery to the olfactory cleft without need of endoscopy or imaging confirmation. In some embodiments, a spray, foam, or liquid spray composition may be delivered in an upright position.

In embodiments, a method of treating ARDS comprises delivery of a composition of the present invention either by intranasal or oral inhaled delivery including inhaler, dry powder inhaler, nebulizer, or direct instillation by spray catheter or liquid.

In embodiments, compositions of the present invention further comprises transplant preservative fluids, such as UW, HTK, or other transplant preservative fluids.

In embodiments, a method of the present invention includes instilling a composition of the present invention into procured organs for transplant for transfer as static cold storage or as a perfusate for ex vivo perfusion in hypothermic conditions or normothermic conditions.

In embodiments, compositions of the present invention further include photosensitizers and are configured for administration along with energy delivery in the form of light, sound, or radiation therapy to treat the chronic manifestations of TBI, SCI, ARDS, and/or Tx organs.

In embodiments, a pharmaceutical manufacturing procedure retains perfluorocarbon such as perflubron for oral or intranasal administration.

In embodiments, a dry powder inhaler composition for direct delivery to lungs of a patient comprises 0-4% (w/w) water; 1-99% of one or more drugs; and an excipient, wherein the composition is a powder comprising particles with a mean diameter of about 0.1 microns to about 5 microns.

In embodiments, a dry powder inhaler composition further comprises one from the group consisting of verteporfin, ribavirin, gemcitabine, aminolevulinate, acetylcysteine, high molecular weight hyaluronan, monoclonal antibodies, tyrosine kinase inhibitors, nintedanib, pirfenidone, and combinations thereof.

In embodiments, a dry powder inhaler composition further comprises checkpoint inhibitors including anti PD-1, anti-PDL1, or CTLA-4 inhibitors, including but not complete listing pembrolizumab, nivolumab, durvalumab, cemiplimab, tremelimumab, retifanlimab, atezolizumab, avelumab, dostarlimab, toripalimab, and combinations thereof.

In embodiments, a dry powder inhaler composition further comprises checkpoint inhibitors including exosomes containing drugs or genetic material such as siRNA or DNA.

In embodiments, a dry powder inhaler composition further comprises an excipient comprising one or more of a sugar, an amino acid, a sugar alcohol, a haloalkane, a self-assembling small molecule, and an inorganic salt.

In embodiments, a dry powder inhaler composition includes a drug at a concentration of >50% (w/w) in the composition.

In embodiments, a dry powder inhaler composition further comprises an excipient comprising mannitol or another sugar alcohol at a ratio of about one part mannitol or another sugar alcohol per ten parts drug by weight.

In embodiments, a dry powder inhaler composition further is configured to be administered with a dry powder inhaler.

In embodiments, a dry powder inhaler composition further comprises an excipient comprising a surfactant.

In embodiments, a dry powder inhaler composition further comprises a surfactant comprising an egg yolk phospholipids and/or polysorbate.

### Examples

### 1. Traumatic Brain Injury

A 24-year-old army infantryman experienced a roadside explosion, and even after 10 minutes, the soldier remained unconsciousness with bilateral reactive pupils but no other severe wounds and had variable hypertensive episodes. (Such variable hypertension is commonly associated with lower rates of being discharged home; see 10.5811/westjem.2022.6.55549.) The earliest examination time at a medical facility was 2½ hrs. (By way of further example, the mean evacuation time was 2.26 hours during war in Croatia; see Hebrang et al. ; Evacuation Times of Civilians and Soldiers Wounded during the War in Croatia, Military Medicine, Volume 171, Issue 11, November 2006, Pages 1045-1050; https://doi.org/10.7205/MILMED.171.11.1045).

In consultation with the medical base who felt the patient experienced a traumatic brain injury, the unit's medic maintained the soldier in supine position with a neck collar and, as instructed, delivered 5 mL of nasal saline spray mist to each nostril followed by 1 mL or 200 mg of intranasal N-acetylcysteine amide and 1 mL or 200 mg of glycine sprays to each nostril. After five minutes, 2 mL spray of neat perflubron was delivered to each nostril and a portable concentrator delivering 67% hydrogen gas (H₂) and 33% oxygen gas (O₂) 33% at 3 lpm. Upon arrival, the patient had a Glasgow Coma Scale (GSC) score of 3 (eyes not open, no verbal or motor response) but with a pupil response.

In a previous study, mild TBI (GCS score of 13-15) was seen in 59.7% of the sample, moderate TBI (GCS score 9-12) in 13.4%, and severe TBI (GCS score <9) in 26.9%. (See doi: 10.1176/appi.neuropsych.21.4.420). In such previous study, 189 patients met the inclusion criteria (GCS 3) and were included in a study. The overall mortality rate was 49.2%. A 6-month follow-up study was available in 82 of 96 survivors; Glasgow Outcome Scale (GOS) scores of 1, 2, 3, and 4 were achieved in 18 (22.0%), 7 (8.5%), 35 (42.7%), and 22 (26.8%) patients, respectively. 13.2% of the entire series achieved a good functional outcome (Glasgow Outcome Scale [GOS] score of 1 or 2).

In contrast, the present nasal instillation therapy is administered and continued for two hours twice daily for two weeks. A non-contrast CT of the head performed after one week is expected to demonstrate an outline of the trauma region by PFOB. The patient is expected to make a remarkable recovery with a good functional outcome GOS of 1-2 at 6 months.

### 2. Spinal Cord Injury

A 54-year-old male commander experienced a T-bone accident and was found to have paralysis from T10-L2 injury. After stabilizing in the emergency room, a CT was performed with findings of a burst fracture and spinal cord injury which was followed by a posterior decompression and stabilization with pedicle screw fixation aiming to relieve the pressure on the spinal cord and restore spinal stability. Using CT guidance, a 5 mL intrathecal catheter instillation into the spinal fluid of sterile perflubron, acetylcysteine, and glycine every 6 hours while inhaling H₂, 67% and O₂, 33% at 3 lpm for 24 hours, then using for 2 hours twice daily.

Note, tissue spinal bridge length predicts outcome in cervical SCI. (See Pfyffer et al.; Prognostic value of tissue bridges in cervical spinal cord injury: a longitudinal, multicenter, retrospective cohort; The Lancet Neurology, Volume 23, Issue 8, 816-825.)

### 3. ARDS

A 36-year-old male, otherwise healthy, chicken farmer who for five days prior presented with cough and fever chills and was diagnosed with H5N1 influenza. The patient developed non-cardiac bilateral chest X-ray infiltrates and worsening hypoxemia with PaO2/FIO2 of 280 while on BIPAP 10/5 cm H2O (note <300 defines ARDS). He received intravenous antibiotics and steroids along with bronchodilator nebulizer treatments every 6 hrs. He has been hemodynamically stable and has mild elevation in WBC 12.3 but significantly elevated C-reactive protein 150.

For comparison, the patients who died from COVID-19 had a tenfold higher level of CRP than the recovered patients, median 100 versus 9.6 milligrams per liter. See doi 10 1002/JMV 26097.

Patient is initiated on H₂ 2.5% plus O₂ 50% by BIPAP for 48 hours until his O₂ saturation could be maintained >90% on a H₂ 67%, O₂ 33% nasal cannula at 6 lpm or less. Further N-acetylcysteine and glycine are also given every six hours. The patient is expected to make a gradual recovery with a steady decline in CRP to less than 50 over the next five days, and H₂ and O₂ at 3 liters per minute are continued for two hours twice a day the next two weeks.

### 4. Transplant Preservative

A 50-year-old diabetic with end stage renal disease on hemodialysis has been awaiting transplant. A donor by death by cardiac death (DCD) became available whose kidney is being delivered using static cold storage, with travel expected to be four hours by air, and the kidney expected to arrive 12 hours after procurement to an OPO to place on an ex vivo pump. The donor hospital used the new technique of placing the kidney in a static cold storage cooler and tubing was hooked to the renal artery and vein and a total of 220 milliliters of a combination of perflubron and UW emulsion solution was added along with NAC 200 mg and glycine 200 mg. The formulation was instilled into a reservoir and thru the UW flushed kidney. The proximal line to the reservoir was then clamped after loading into the reservoir and kidney and an H₂ 67%/O₂ 33% concentrator was used to deliver 1-3 liters per minute into the 110-milliliter reservoir (the reservoir fluid was in fluid contact with the 110 mL in the kidney vasculature), and the gas traversed the kidney and exited the renal vein expelling excess gas plus CO₂. The H₂ and O₂ coupled with the NAC and glycine were expected to resolve any reactive oxygen species (ROS), enabling a better preservative solution resulting in a qualified donor organ and with a decreased incidence of postoperative graft dysfunction.

Note, according to the National Kidney Foundation, 71% of donor kidneys are preserved in static cold storage in 27,332 kidney transfer plants were performed in 2023 with 90,323 people on the waiting list. Global health and Wellness Feb. 6, 2025. “For kidney, liver, heart, lung and intestine, 24%, 29%, 58%,72%, and 98% of donor organs are respectively wasted.” (See Liu et al.; Organ Preservation: History, Advancements, and Perspectives; Engineering, volume 44, 2025, pages 112-134, ISSN 2095-8099;

https://doi.org/10.1016/j.eng.2024.12.020). “Our cohort comprised 173,792 deceased donor kidneys procured for transplantation during the study period. The overall hypothermic machine perfusion rate was 39.1% (n=67,965). The overall kidney discard rate was 20.9% (n=36,242). The discard rate for pumped kidneys was 20.2% (n=13,755), while static cold storage kidneys had a discard rate of 21.2% (n=22,487).” (See Ovdat et al.; 246.3: Regional disparities in hypothermic machine perfusion pump utilization and kidney discard rates in the United States; Transplantation 108(9S): September 2024; DOI: 10.1097/01.tp.0001064752.39861.de).
5. Correlation of eNO with Viral Load

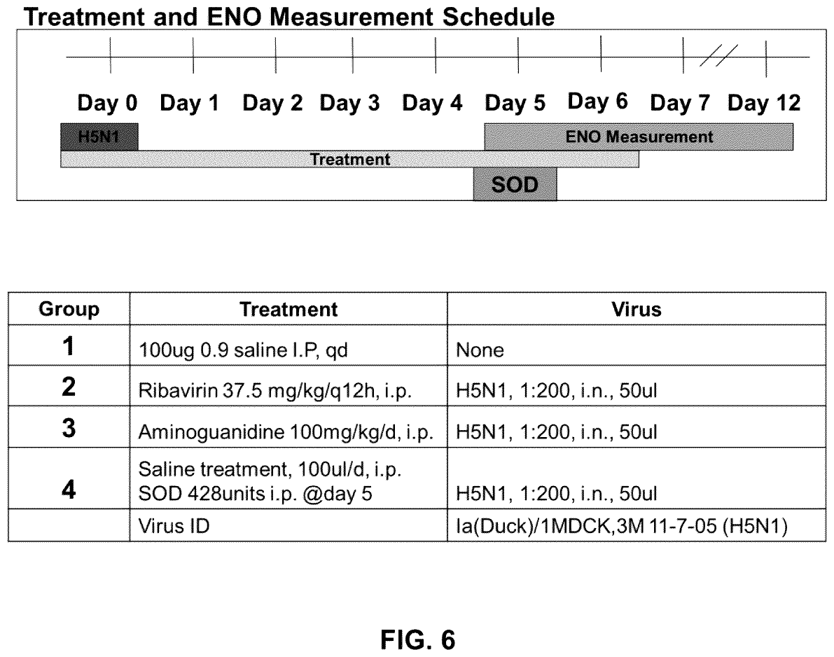

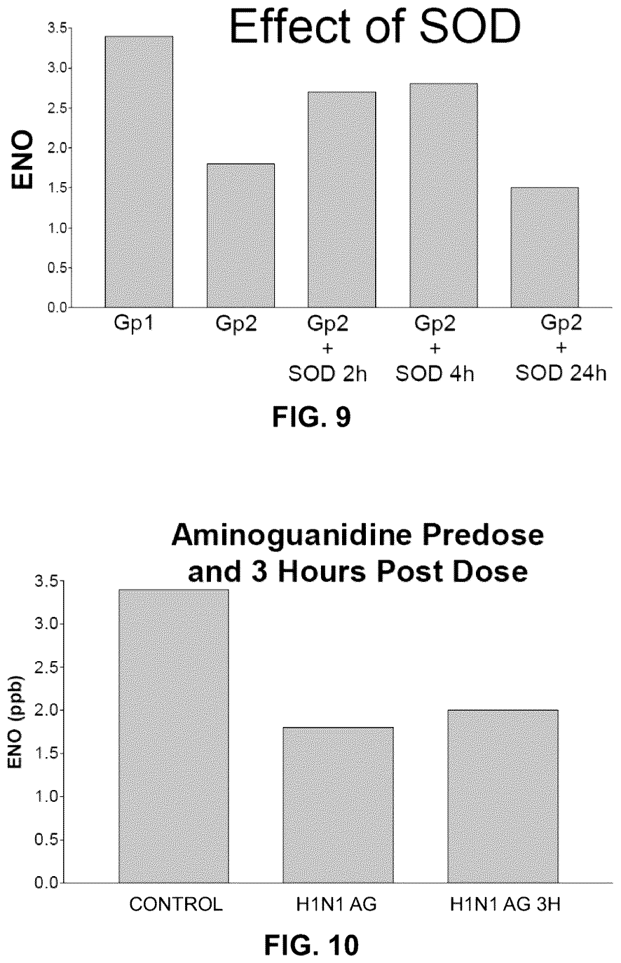

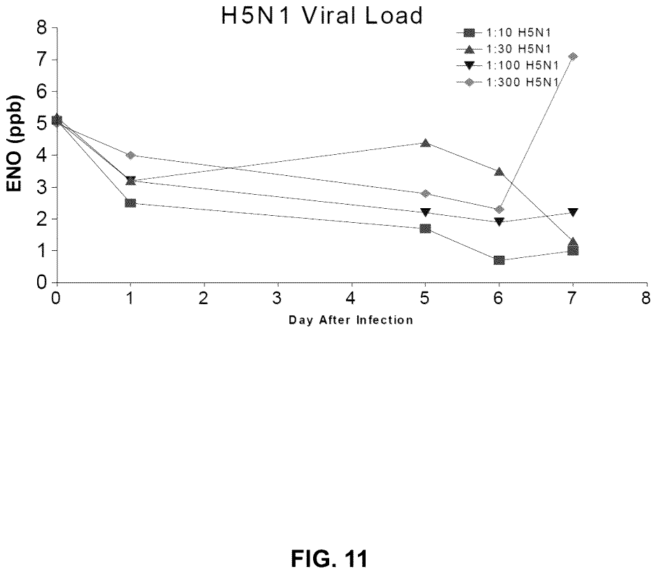

Exhaled nitric oxide (eNO) levels were observed to enable the discriminating between normal, H5N1 infected and treated H5N1 infected mice, as further shown in FIGS. 6-13.

eNO was measured in mice from the following control and experimental groups: normal, uninfected; infected with H5N1; infected with H5N1 and treated with aminoguanidine; and infected with H5N1 and treated with Ribavirin, each to observe eNO in response to viral load. The treatment and eNO measurement schedule and drug treatment groups and control are depicted in FIG. 6.

The experiment used seven mice per box (least survival rate among groups at D5). The initial equilibration time based on tidal volume (1 ml/min/gram). Data was recorded every 5 minutes. The experimental data points were taken at equilibration at 16-23 minutes. The eNO was measured by chemiluminescence (Aerocrine Niox).

Animals were infected with H5N1 (Duck) Influenza virus at day 0 (D0), (1:200 , 50 ul, i.n.). eNO was measured using a NIOX chemiluminescence analyzer (Aerocrine, Sweden) and adapted to obtain measurements from a 2-liter chamber. eNO levels were allowed to build in the chamber for 20 minutes while nitric oxide (NO)-free air was drawn through the box at a rate of 5 ml/min. Initially 10 mice/box were measured, but due to death in G4, all subsequent measurements used 7 mice/box. eNO measurements were performed at D5 p.i. Treatment groups. G1-control. No infection or treatment G2-H5N1/aminoguanidine (100 mg/kg/d) G3—H5N1/Ribavirin (37.5 mg/kg/q12h) and G4—H5N1/saline (100 ul/d, i.p).

eNO was reproducibly measured in mice. H5N1 infected mice decreased eNO compared to control mice. SOD partially attenuated the change in eNO. Aminoguanidine and ribavirin treated animals had lower levels of eNO. eNO appears to correlate to viral load.

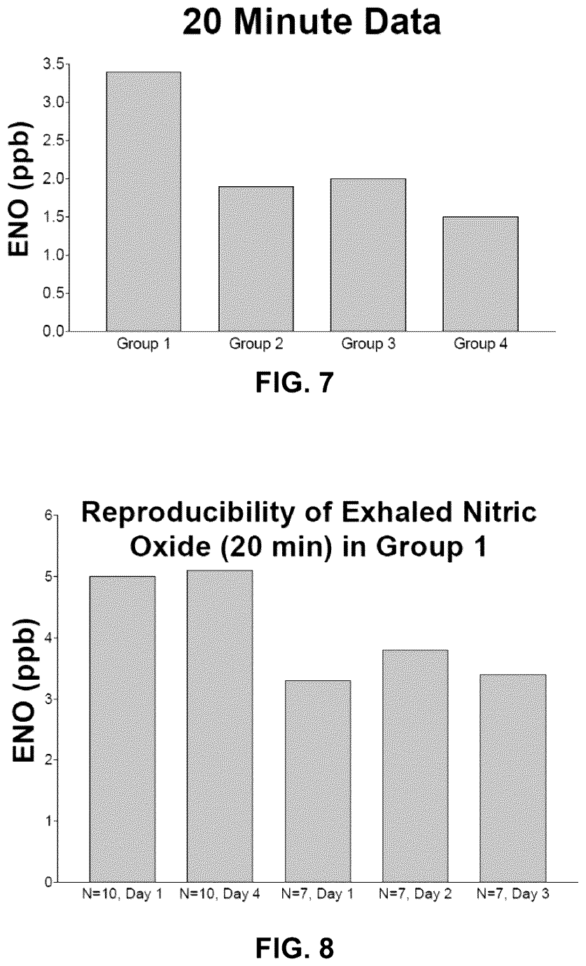

eNO levels over 4 days at 20 minutes remained constant. D4 and D7 p.i. (n=10) were 5.0 and 5.1 ppb, respectively. D4, D5 and D6 p.i. (n=7) were 3.8, 3.3 and 3.4 ppb, respectively. Treatment Groups at D5p.i.: G1=3.3 ppb, G2=1.9 ppb, 3=2.0 ppb, and G4=1.5 ppb. See FIGS. 7-11.

ENO levels in H5N1 mice was unexpectedly decreased. It is generally known in the art that superoxide dismutase (SOD) scavenges superoxide and may attenuate decrease in eNO in response to oxidative stress. eNO may have further potential to monitor clinical response to therapy, to rapidly screen treatments for human influenza, and to be an epidemiological tool.

eNO in ribavirin treated animals infected with H5N1 follow a similar course to human volunteers infected with a conventional influenza virus as reported in the literature.

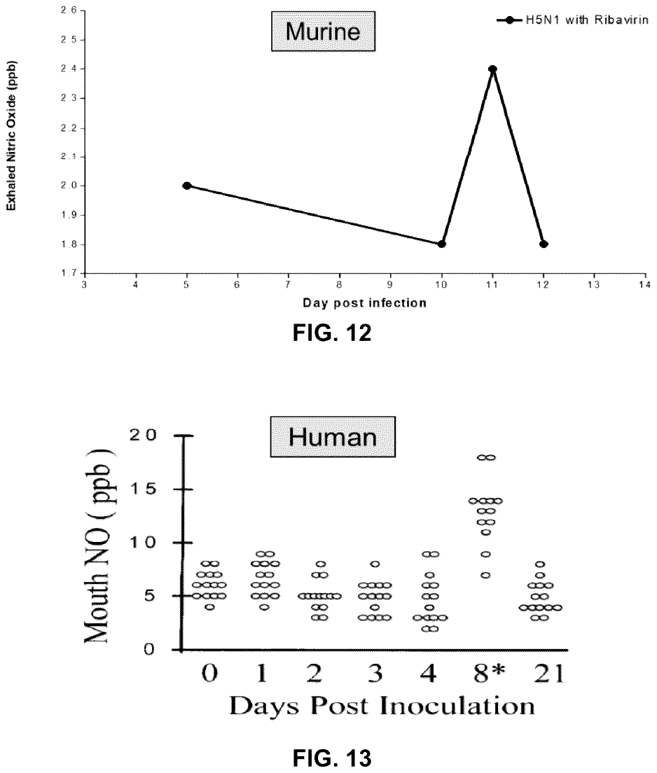

Compare FIG. 12 and FIG. 13.

An increase in eNO was anticipated in H5N1 infected mice compared to normal controls. However, a decrease in eNO was actually observed in the H5N1 infected mice, and this was postulated to be due to oxidative stress (NO binding with superoxide). Therefore, G4 mice at D5 were treated with bovine superoxide dismutase (SOD) (7 mg/kg, i.p.). eNO at pre-dose, 2 h, 4 h, and 24 h post-SOD were 1.5, 2.7, 2.8 and 1.5 ppb respectively and supported the aforementioned theory. Since ribavirin has also been shown to reduce nitric oxide synthetase the reduced eNO was not unexpected.

eNO was reproducibly measured in mice. H5N1 infected mice decreased eNO compared to control mice. SOD partially attenuated the change in eNO. Aminoguanidine and ribavirin treated animals had lower levels of eNO. eNO appears likely to correlate to viral load.

Certain terminology is used in the description for convenience in reference only and will not be limiting. For example, up, down, front, back, right, and left refer to the invention as orientated in the view being referred to. The words “inwardly” and “outwardly” refer to directions toward and away from, respectively, the geometric center of the aspect being described and designated parts thereof. Forwardly and rearwardly are generally in reference to the direction of travel, if appropriate. Additionally, anatomical terms are given their usual meanings. For example, proximal means closer to the trunk of the body, and distal means further from the trunk of the body. Said terminology shall include the words specifically mentioned, derivatives thereof, and words of similar meaning.

As used in this specification and the appended claims, the singular forms “a”, “an”, and “the” include plural references unless the context clearly dictates otherwise. Thus, for example, a reference to “a method” includes one or more methods, elements, and/or steps of the type described herein and/or which will become apparent to those persons skilled in the art upon reading this disclosure and so forth.

As used in this specification and the appended claims, the use of the term “about” means a range of values including and within 15% above and below the named value, except for nominal temperature. For example, the phrase “about 3 mM” means within 15% of 3 mM, or 2.55-3.45, inclusive. Likewise, the phrase “about 3 millimeters (mm)” means 2.55 mm-3.45 mm, inclusive. When temperature is used to denote change, the term “about” means a range of values including and within 15% above and below the named value. For example, “about 5° C.,” when used to denote a change such as in “a thermal resolution of better than 5° C. across 3 mm,” means within 15% of 5° C., or 4.25° C.-5.75° C. When referring to nominal temperature, such as “about −50° C. to about +50° C.,” the term “about” means +5° C. Thus, for example, the phrase “about 37° C.” means 32° C.-42° C.

Unless defined otherwise, all technical and scientific terms used herein have the same meaning as commonly understood by one of ordinary skill in the art to which this invention belongs. Although any systems, elements, methods and materials similar or equivalent to those described herein can be used in the practice or testing of the present invention, the preferred systems, elements, and methods and materials are now described. All publications mentioned herein are incorporated herein by reference to describe in their entirety.

“Substantially” means to be more-or-less conforming to the particular dimension, range, shape, concept, or other aspect modified by the term, such that a feature or component need not conform exactly. For example, a “substantially cylindrical” object means that the object resembles a cylinder but may have one or more deviations from a true cylinder. “Comprising,” “including,” and “having” (and conjugations thereof) are used interchangeably to mean including but not necessarily limited to, and are open-ended terms not intended to exclude additional, unrecited elements or method steps.

Changes may be made in the above methods, devices and structures without departing from the scope hereof. Many different arrangements of the various components depicted, as well as components not shown, are possible without departing from the spirit and scope of the present invention. Embodiments of the present invention have been described with the intent to be illustrative and exemplary of the invention, rather than restrictive or limiting of the scope thereof. Alternative embodiments will become apparent to those skilled in the art that do not depart from its scope. Specific structural and functional details disclosed herein are not to be interpreted as limiting, but merely as a representative basis for teaching one of skill in the art to employ the present invention in any appropriately detailed structure. A skilled artisan may develop alternative means of implementing the aforementioned improvements without departing from the scope of the present invention.

It will be understood that certain features and subcombinations are of utility and may be employed without reference to other features and subcombinations and are contemplated within the scope of the claims. Not all steps listed in the various figures need be carried out in the specific order described.

It is also to be understood that the following claims are intended to cover all of the generic and specific features of the invention herein described, and all statements of the scope of the invention which, as a matter of language, might be said to fall therebetween.

Having thus described the invention, what is claimed as new and desired to be secured by Letters Patent is:

1. A pharmaceutical composition comprising:
a perfluorocarbon;
0.5% to 4% (w/w) hydrogen gas;
10% to 96% (w/w) oxygen gas;
N-acetylcysteine or N-acetylcysteine amide; and
glycine.

2. The composition of claim 1, wherein:
said perfluorocarbon comprises perflubron.

3. The composition of claim 2, wherein:
said perflubron is in the amount of 0.1 mL to 20 mL.

4. The composition of claim 1, wherein:
said N-acetylcysteine or N-acetylcysteine amide comprises N-acetylcysteine amide in the amount of 100 mg/mL to 600 mg/mL.

5. The composition of claim 1, wherein:
said glycine is in the amount of 100 mg/mL to 600 mg/mL.

6. The composition of claim 1, further comprising:
catalase.

7. The composition of claim 1, wherein:
said composition is one selected from the group consisting of: a dry powder, a liquid, a foam, an aerosol, a hydrogel, and an emulsion.

8. The composition of claim 1, wherein:
said composition is configured for delivery to a target tissue by one selected from the group consisting of: a syringe, an inhaler, and a spray catheter.

9. The composition of claim 1, further comprising:
one selected from the group consisting of: verteporfin, ribavirin, gemcitabine, aminolevulinate, acetylcysteine high molecular weight hyaluronan, monoclonal antibodies, tyrosine kinase inhibitors, nintedanib, pirfenidone, exosomal siRNA, exosomal DNA, IRDye700DX, IRDye800CW, indocyanine green (ICG), and combinations thereof.

10. The composition of claim 1, further comprising:
a transplant preservative fluid.

11. The composition of claim 1, wherein:
said composition comprises a dry powder composition comprising 0 to 4% (w/w) water and having particles with a mean diameter of 0.1 microns to 5 microns.

12. The composition of claim 1, further comprising:
a checkpoint inhibitor selected from the group consisting of: pembrolizumab, nivolumab, durvalumab, cemiplimab, tremelimumab, retifanlimab, atezolizumab, avelumab, dostarlimab, toripalimab, and combinations thereof.

13. The composition of claim 1, further comprising:
an excipient selected from the group consisting of: a sugar, an amino acid, a sugar alcohol, a haloalkane, a self-assembling small molecule, an inorganic salt, a surfactant, and combinations thereof.

14. The composition of claim 1, further comprising:
an emulsifier.

15. A method of treating a medical condition comprising:
administering a perfluorocarbon, N-acetylcysteine or N-acetylcysteine amide, and glycine to a target tissue; and
administering hydrogen gas and oxygen gas to said target tissue.

16. The method of claim 15, wherein:
said medical condition is one selected from the group consisting of: traumatic brain injury, spinal cord injury, acute respiratory distress syndrome (ARDS), and transplant ischemia and dysfunction.

17. The method of claim 15, wherein:
said administering steps comprise intranasal administration of said perfluorocarbon, N-acetylcysteine or N-acetylcysteine amide, and glycine and intranasal administration of said hydrogen gas and said oxygen gas.

18. The method of claim 17, further comprising:
positioning a patient in supine position to enable delivery of said perfluorocarbon, N-acetylcysteine or N-acetylcysteine amide, glycine, hydrogen gas, and oxygen gas to an olfactory cleft of said patient.

19. The method of claim 15, wherein:
said administering steps comprise instilling said perfluorocarbon, N-acetylcysteine or N-acetylcysteine amide, and glycine into a procured organ for transplant and instilling said hydrogen gas and said oxygen gas into said procured organ for transfer as static cold storage or as a perfusate for ex vivo perfusion in hypothermic conditions.

20. The method of claim 15, further comprising:
measuring exhaled nitric oxide (eNO) in a patient to determine severity of reactive oxygen species (ROS).
